(19) United States
(12) Patent Application Publication
(10) Pub. No.: US 2023/0201567 A1
(43) Pub. Date: Jun. 29, 2023

(54) MUSCLE STIMULATION ASSEMBLY

(71) Applicant: Debbra Jones, Windsor, NC (US)

(72) Inventor: Debbra Jones, Windsor, NC (US)

(21) Appl. No.: 17/564,310

(22) Filed: Dec. 29, 2021

(51) Int. Cl. A61N 1/04; A61N 1/36

(52) U.S. Cl. CPC A61N 1/0452; A61N 1/0484; A61N 1/3603

(57) ABSTRACT

A muscle stimulation assembly includes a plurality of sleeves that is each wearable on a selected limb on a user's body. Each of the sleeves is comprised of a resiliently stretchable material thereby facilitating the sleeves to compress around the selected limb. A plurality of conduction arrays is each integrated into a respective one of the sleeves. Moreover, the conduction array in the respective sleeve engages skin on the limb when the respective sleeve is worn and is in electrical communication with muscles in the limb when the respective sleeve is worn. A control system is placed into selective electrical communication with the conduction array in the respective sleeve when the respective sleeve is worn. The control system generates an electrical impulse and communicates the electrical impulse to the conduction array in the respective sleeve to flex muscles in the limb for therapeutic purposes.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to stimulation devices and more particularly pertains to a new stimulation device for electrical muscle stimulation therapy.

### (2) Description of Related Art Including Information Disclosed Under 37 CFR 1.97 and 1.98

The prior art relates to stimulation devices including a compression garment that is wearable around a user's limb and a plurality of electrical contacts that are integrated into the compression garment. Additionally, a feedback sensor is included that is in electrical communication with the electrical contacts for facilitating therapeutic radiation to a body part.

### BRIEF SUMMARY OF THE INVENTION

An embodiment of the disclosure meets the needs presented above by generally comprising a plurality of sleeves that is each wearable on a selected limb on a user's body. Each of the sleeves is comprised of a resiliently stretchable material thereby facilitating the sleeves to compress around the selected limb. A plurality of conduction arrays is each integrated into a respective one of the sleeves. Moreover, the conduction array in the respective sleeve engages skin on the limb when the respective sleeve is worn and is in electrical communication with muscles in the limb when the respective sleeve is worn. A control system is placed into selective electrical communication with the conduction array in the respective sleeve when the respective sleeve is worn. The control system generates an electrical impulse and communicates the electrical impulse to the conduction array in the respective sleeve to flex muscles in the limb for therapeutic purposes.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a muscle stimulation assembly according to an embodiment of the disclosure.

FIG. 2 is a front view of a sleeve of an embodiment of the disclosure.

FIG. 3 is a cross sectional view taken along line 3-3 of FIG. 2 of an embodiment of the disclosure.

FIG. 4 is an exploded perspective view of an input port of an embodiment of the disclosure.

FIG. 5 is a perspective in-use view of an embodiment of the disclosure.

FIG. 6 is a schematic view of an embodiment of the disclosure.

FIG. 7 is a perspective view of a control housing of an embodiment of the disclosure.

### DETAILED DESCRIPTION OF THE INVENTION

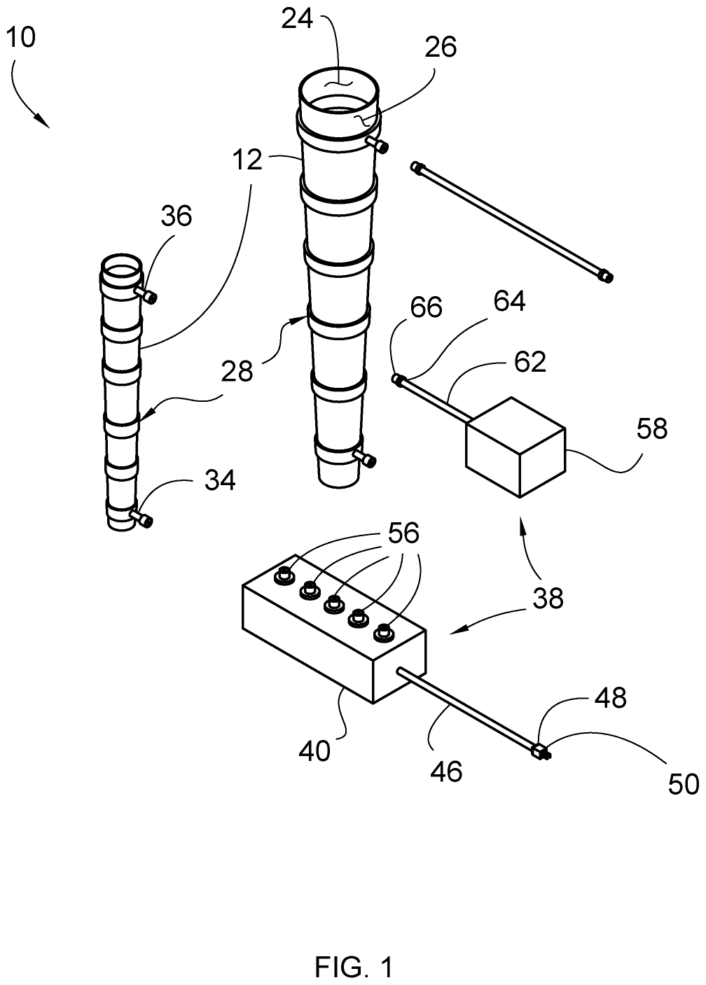

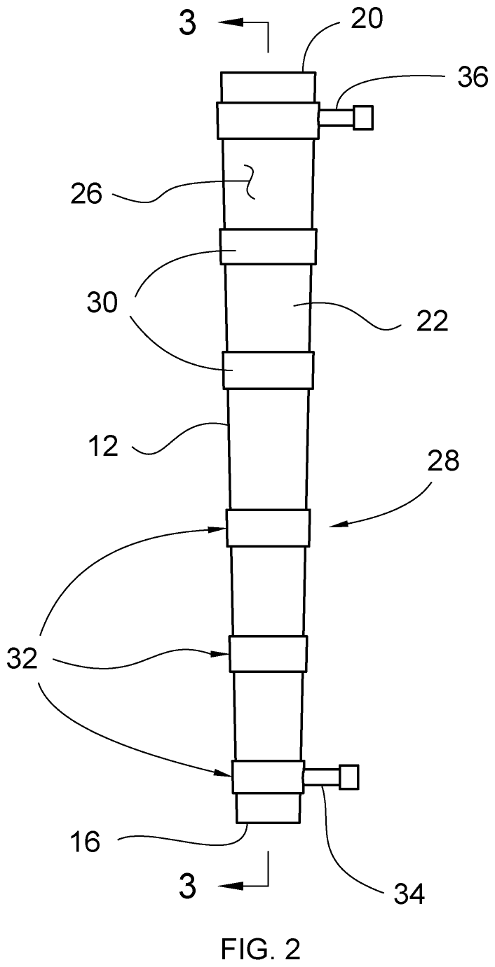

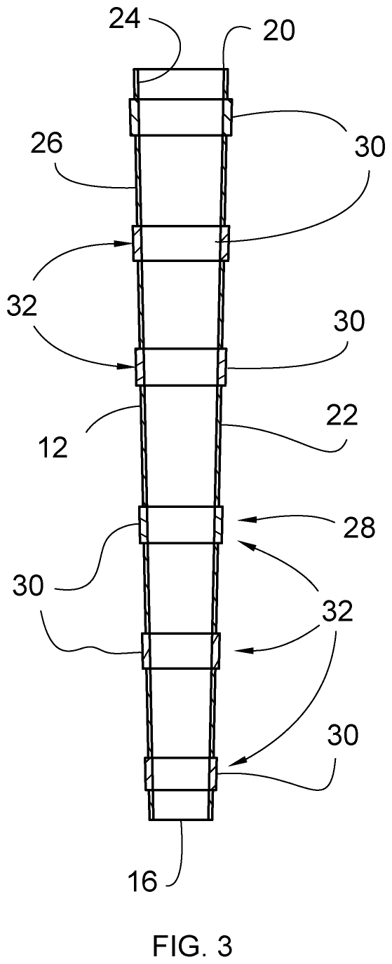

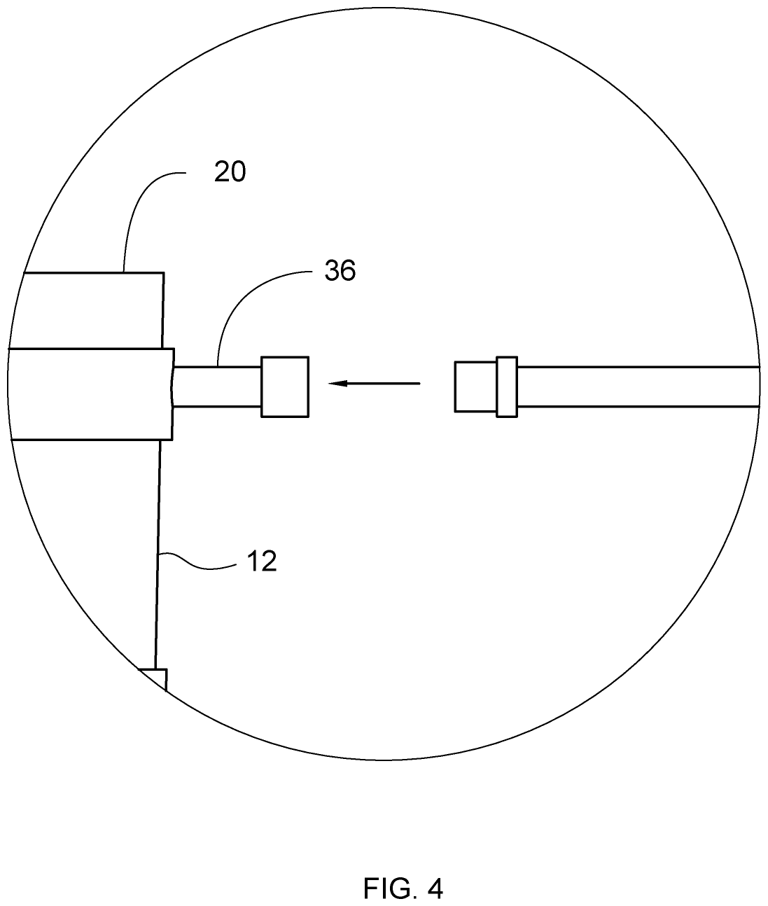

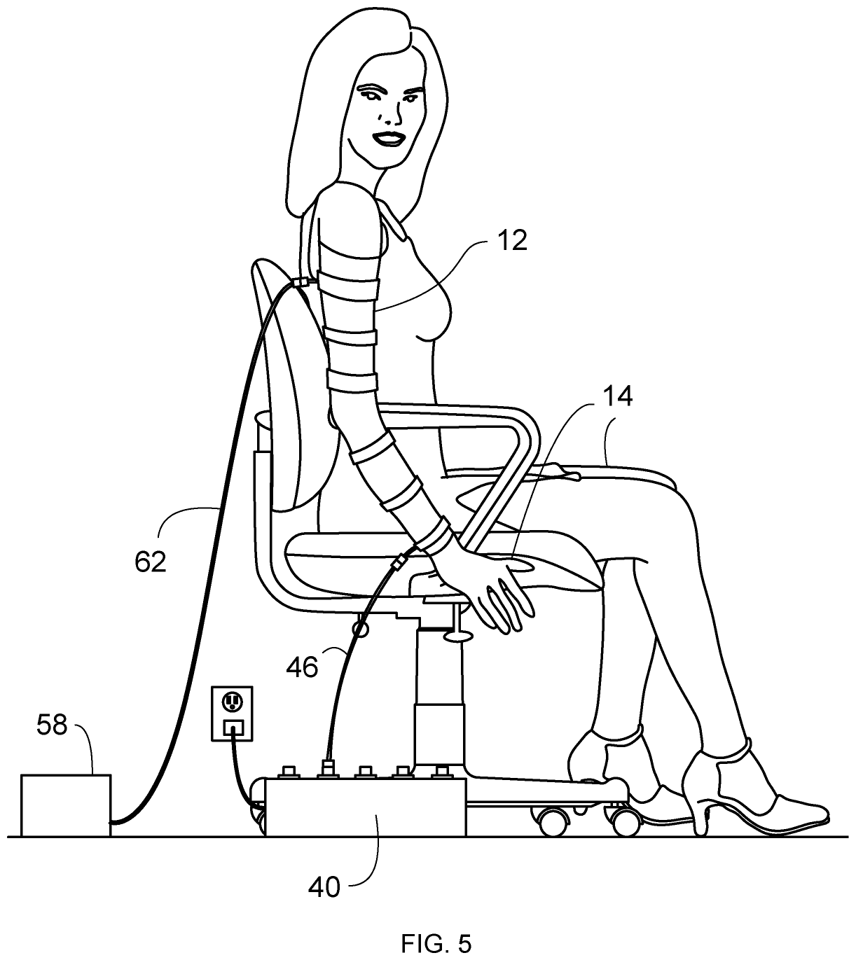

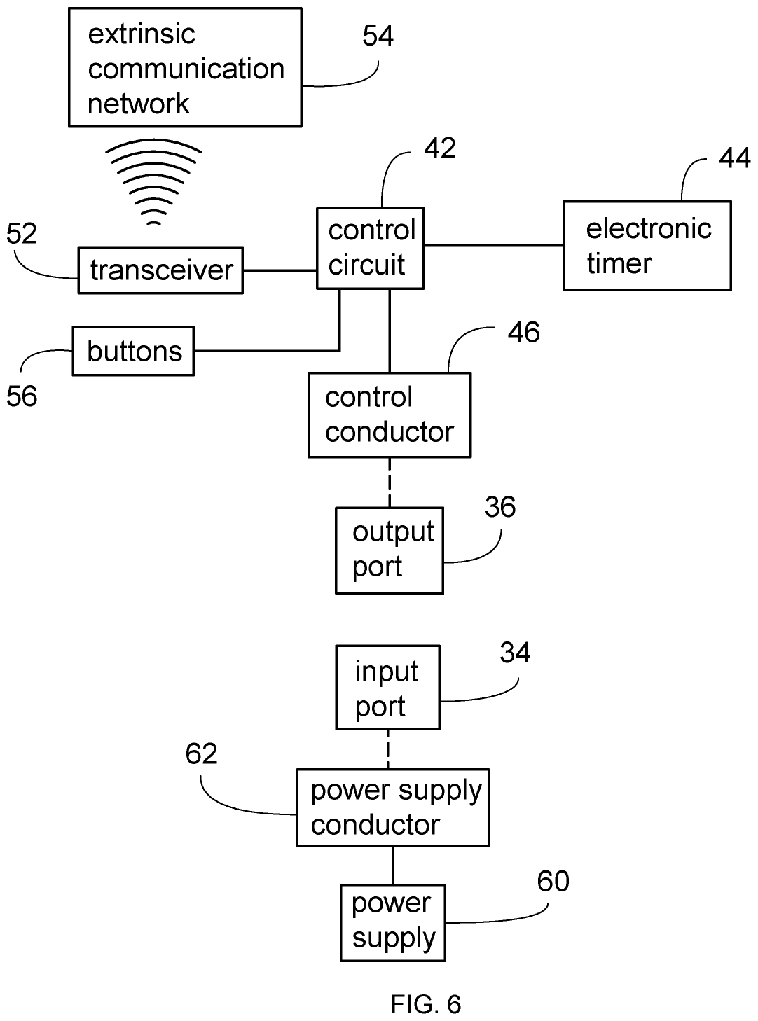

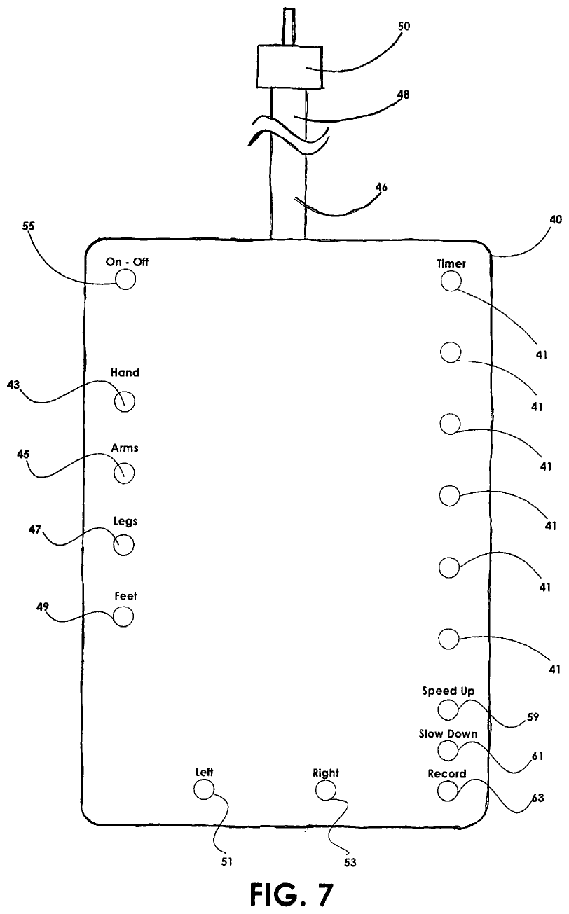

With reference now to the drawings, and in particular to FIGS. 1 through 7 thereof, a new stimulation device embodying the principles and concepts of an embodiment of the disclosure and generally designated by the reference numeral 10 will be described.

As best illustrated in FIGS. 1 through 7, the muscle stimulation assembly 10 generally comprises As best illustrated in FIGS. 1 through 6, the muscle stimulation assembly 10 generally comprises a plurality of sleeves 12 that is each wearable on a selected limb 14 on a user's body. The user may be a physically disabled user that has lost or limited use of the user's limbs due to spinal injury, stroke or illness. Each of the sleeves 12 is comprised of a resiliently stretchable material thereby facilitating the sleeves 12 to compress around the selected limb 14. Each of the sleeves 12 has a first end 16, a second end 20 and an outer wall 22 extending therebetween, and the outer wall 22 has an inner surface 24 and an outer surface 26. Additionally, the sleeves 12 may be worn around the user's torso, the user's hands and the user's feet.

A plurality of conduction arrays 28 is provided and each of the conduction arrays 28 is integrated into a respective one of the sleeves 12. Each of the conduction arrays 28 is comprised of an electrically conductive material. Moreover, the conduction array 28 in the respective sleeve 12 engages skin on the limb 14 when the respective sleeve 12 is worn. In this way the conduction array in the respective sleeve 12 is in electrical communication with muscles in the limb 14 when the respective sleeve 12 is worn.

Each of the conduction arrays 28 comprises a plurality of conductors 30 that is integrated into the outer wall 22 of the respective sleeve 12. Each of the conductors 30 is in electrical communication with the limb 14 when the respective sleeve 12 is worn. Moreover, the conductors 30 are arranged in the respective sleeve 12 to define a plurality of rings 32 each extending around an entire circumference of the respective sleeve 12. The rings 32 are spaced apart from each other and are distributed between the first end 16 and the second end 20 of the respective sleeve 12. The conductors 30 are further arranged to extend between each of the rings 32 such that each of the rings 32 within the respective sleeve 12 is in electrical communication with each other.

An input port 34 is coupled to the outer wall 22 of the respective sleeve 12 and the input port 34 is positioned proximate the first end 16 of the sleeve. The input port 34 is electrically coupled to an adjacent one of the rings 32. An output port 36 is coupled to the outer wall 22 of the respective sleeve 12 and the output port 36 is positioned proximate the second end 20 of the sleeve. The output port 36 is electrically coupled to an adjacent one of the rings 32 to complete a circuit between the output port 36, each of the rings 32 and the input port 34.

A control system 38 is provided and the control system 38 is placed into selective electrical communication with the conduction array 28 in the respective sleeve 12 when the respective sleeve 12 is worn. The control system 38 generates an electrical impulse and communicates the electrical impulse to the conduction array 28 in the respective sleeve 12. The conduction array 28 in the respective sleeve 12 communicates the electrical impulse to nerves in the limb 14. In this way the control system 38 causes muscles in the limb 14 to flex for therapeutic purposes.

The control system 38 comprises a control housing 40 and a control circuit 42 that is positioned within the control housing 40. The control circuit 42 stores data comprising a plurality of operational commands with respect to the electrical impulse. An electronic timer 44 is positioned within the control housing 40 and the electronic timer 44 is electrically coupled to the control circuit 42. The electronic timer 44 is actuatable to count down a variety of pre-determined durations of time, which may range between approximately 5.0 seconds and 60.0 minutes.

A control conductor 46 is coupled to and extends away from the control housing 40 and the control conductor 46 is electrically coupled to the control circuit 42. The control conductor 46 has a distal end 48 with respect to the control housing 40 a control plug 50 is electrically coupled to the distal end 48. The control plug 50 is pluggable into the output port 36 in the respective sleeve 12 such that the control circuit 42 is in electrical communication with the conductors 30 in the respective sleeve 12. A plurality of the control conductors 46 may be provided thereby facilitating a plurality of the sleeves 12 to be electrically coupled to the control circuit 42.

A transceiver 52 is positioned within the control housing 40 and the transceiver 52 is electrically coupled to the control circuit 42. The transceiver 52 is in electrical communication with an extrinsic communication network 54, such as a wifi internet router or the like. In this way the control circuit 42 can be remotely monitored and controlled via an app on a smart phone or the like. The transceiver 52 may be a radio frequency transceiver or the like and the transceiver 52 may employ a WPAN signal and Bluetooth communication protocols.

A plurality of buttons 56 is each movably coupled to the control housing 40. Each of the buttons 56 is electrically coupled to the control circuit 42. Each of the buttons 56 controls operational parameters of the control circuit 42 and the transceiver 52. The plurality of buttons 56 includes a plurality of timer buttons 41 that is each assigned to a respective one of the variety of durations of time associated with the electronic timer 44. The electronic timer 44 counts down a respective one of the durations of time when the respective timer button 41 is depressed. Furthermore, the control circuit 42 is turned off when the electronic timer 44 completes counting down the respective duration of time.

The plurality of buttons 56 includes a hand button 43 and a respective one of the conduction arrays 28 in a respective one of the sleeves 12 is actuated when the hand button 43 is depressed such that the respective sleeve stimulates nerves associated with the user's hand. In this way muscles that are associated with hand movement are stimulated for inhibiting atrophy in the user's hand. The plurality of buttons 56 includes an arm button 45 and a respective one of the conduction arrays 28 in a respective one of the sleeves 12 is actuated when the arm button 45 is depressed. In this way the respective sleeve 12 stimulates nerves in the user's arm. Thus, muscles in the user's upper arm and forearm are stimulated for inhibiting atrophy in the user's arm.

The plurality of buttons 56 includes a leg button 47 and a respective one of the conduction arrays 28 in a respective one of the sleeves 12 is actuated when the leg button 47 is depressed such that the respective sleeve 12 stimulates nerves in the user's leg. In this way the respective sleeve 12 stimulates nerves in the user's leg thereby facilitating muscles in the user's leg to be stimulated to inhibit atrophy in the user's leg. The plurality of buttons 56 includes a foot button 49 and a respective one of the conduction arrays 28 in a respective one of the sleeves 12 is actuated when the foot button 49 is depressed. In this way the respective sleeve 12 stimulates nerves associated with the user's foot such that muscles associated with foot movement are stimulated for inhibiting atrophy in the user's foot.

The plurality of buttons 56 includes a left button 51 and each of the conduction arrays 28 in each of the sleeves 12 that is worn on the user's left arm and left leg is actuated when the left button 51 is depressed. Furthermore, the sleeves 12 that are worn on the user's right arm and right leg are not actuated when the left button 51 is depressed. The plurality of buttons 56 includes a right button 53 and each of the conduction arrays 28 in each of the sleeves 12 is worn on the user's right arm and right leg is actuated when the right button 53 is depressed. Moreover, the sleeves 12 that are worn on the user's left arm and left leg are not actuated when the right button 53 is depressed. Additionally, the plurality of buttons 56 includes a power button 55 and the control circuit 42 is turned on or off, respectively, when the power button 55 is depressed.

The plurality of buttons 56 includes a speed increase button 59, a speed decrease button 61, and a record button 63. The speed increase button 59 and the speed decrease button 61 alter a speed of oscillations in the electrical impulse. Each of the sleeves 12 can be worn on a respective limb 14 and each of the sleeves 12 can be assigned to a respective one of the hand button 43, the arm button 45, the leg button 47 and the foot button 49. Additionally, the sleeves 12 may be assigned to a respective one of the left button 51 and right buttons 53. The record button 63 can be depressed thereby facilitating a sequence of electrical impulses to be programmed into the control circuit 42 to define an operational sequence. The operational sequence may be determined by a medical care professional or other individual responsible for the user's physical rehabilitation. Furthermore, the spacing of the rings 32 facilitates different muscle groups to be specifically targeted for electrical stimulation therapy. In this way, only muscles in the user's upper arm, for example, could be targeted for electrical stimulation when the arm button 45 is depressed. Additionally, only muscles in the user's lower arm could be targeted for electrical stimulation when the hand button 43 is depressed. Moreover, various sequences of rings 32 can be actuated and de-actuated in accordance with the operational sequence thereby facilitating specific targeting of muscle groups for physical rehabilitation.

The control system 38 includes a power supply housing 58, and a power supply 60 is positioned within the power supply housing 58. The power supply 60 may comprise a rechargeable battery or the like and a transformer. Additionally, the power supply 60 may include a power cord that can be plugged into a female electrical outlet or other power source. The power supply 60 may be a solid state power supply and the power supply 60 may be a power supply common to electrical muscle stimulation units.

A power supply conductor 62 is coupled to and extends away from the power supply housing 58. The power supply conductor 62 is electrically coupled to the power supply 60 and the power supply conductor 62 has a distal end 64 with respect to the power supply housing 58. A plug 66 is electrically coupled to the distal end 64 of the power supply conductor 62 and the plug 66 on the power supply conductor 62 is pluggable into the input port 34 in the respective sleeve 12. Thus, the power supply 60 is in electrical communication with the control circuit 42 when the plug 66 on the control conductor 46 is plugged into the output port 36. In this way the power supply 60 may generate the electrical impulse in accordance with the operational commands stored in the control circuit 42. A plurality of power supply conductors 62 may be provided thereby facilitating a plurality of the sleeves 12 to be electrically coupled to the power supply 60.

In use, selected ones of the sleeves 12 are worn on respective limbs 14 for physical therapy to reduce muscle atrophy due to musculoskeletal injuries, stroke, and other injuries that can limit or eliminate physical mobility. Each of the sleeves 12 is plugged into the control conductor 46 and the power supply conductor 62. The power supply 60 sends the electrical impulse to the conductor array in each of the sleeves 12 in accordance to settings determined with the buttons 56 on the control housing 40 or with a program stored in the control circuit 42. Thus, the conductor array in each of the sleeves 12 stimulates the muscles in the respective limb 14 for electrical muscle stimulation. Respective ones of the hand button 43, the arm button 45, the leg button 47, and the foot button 49 are depressed in accordance to the user's preference regarding the specific group of muscles that are intended to receive physical therapy. In this way the user can exercise complete control over which limbs receive therapy as well as being able to control the intensity and duration of time of the therapy.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A muscle stimulation assembly being worn on a user's limbs wherein said assembly is configured to electrically stimulate the limbs for rehabilitative purposes, said assembly comprising:
a plurality of sleeves, each of said sleeves being wearable on a selected limb on a user's body, each of said sleeves being comprised of a resiliently stretchable material thereby facilitating said sleeves to compress around the selected limb;
a plurality of conduction arrays, each of said conduction arrays being integrated into a respective one of said sleeves, each of said conduction arrays being comprised of an electrically conductive material, said conduction array in said respective sleeve engaging skin on the limb when said respective sleeve is worn wherein said conduction array in said respective sleeve is configured to be in electrical communication with muscles in the limb when said respective sleeve is worn; and
a control system being placed into selective electrical communication with said conduction array in said respective sleeve when said respective sleeve is worn, said control system generating an electrical impulse and communicating said electrical impulse to said conduction array in said respective sleeve, said conduction array in said respective sleeve communicating the electrical impulse to nerves in the limb wherein said control system is configured to flex muscles in the limb for therapeutic purposes.

2. The assembly according to claim 1, wherein:
each of said sleeves has a first end, a second end and an outer wall extending therebetween, said outer wall having an inner surface and an outer surface; and
each of said conduction arrays comprises a plurality of conductors being integrated into said outer wall of said respective sleeve wherein each of said conductors is configured to be in electrical communication with the limb when said respective sleeve is worn.

3. The assembly according to claim 2, wherein said conductors are arranged in said respective sleeve to define a plurality of rings each extending around an entire circumference of said respective sleeve, said rings being spaced apart from each other and being distributed between said first end and said second end of said respective sleeve, said conductors being further arranged to extend between each of said rings such that each of said rings within said respective sleeve is in electrical communication with each other.

4. The assembly according to claim 3, further comprising an input port being coupled to said outer wall of said respective sleeve, said input port being positioned proximate said first end of said sleeve, said input port being electrically coupled to an adjacent one of said rings.

5. The assembly according to claim 4, further comprising an output port being coupled to said outer wall of said respective sleeve, said output port being positioned proximate said second end of said sleeve, said output port being electrically coupled to an adjacent one of said rings.

6. The assembly according to claim 5, wherein said control system comprises:
a control housing; and
a control circuit being positioned within said control housing, said control circuit storing data comprising a plurality of operational commands with respect to said electrical impulse.

7. The assembly according to claim 6, further comprising a control conductor being coupled to and extending away from said control housing, said control conductor being electrically coupled to said control circuit, said control conductor having a distal end with respect to said control housing.

8. The assembly according to claim 7, wherein said distal end has a control plug being electrically coupled thereto, said control plug being pluggable into said output port in said respective sleeve such that said control circuit is in electrical communication with said conductors in said respective sleeve.

9. The assembly according to claim 6, further comprising a transceiver being positioned within said control housing, said transceiver being electrically coupled to said control circuit, said transceiver being in electrical communication with an extrinsic communication network.

10. The assembly according to claim 9, further comprising a plurality of buttons, each of said buttons being movably coupled to said control housing, each of said buttons being electrically coupled to said control circuit, each of said buttons controlling operational parameters of said control circuit and said transceiver.

11. The assembly according to claim 8, wherein said control system comprises:
a power supply housing; and
a power supply being positioned within said power supply housing.

11. The assembly according to claim 10, further comprising a power supply conductor being coupled to and extending away from said power supply housing, said power supply conductor being electrically coupled to said power supply, said power supply conductor having a distal end with respect to said power supply housing.

12. The assembly according to claim 11, wherein said distal end of said power supply conductor has a plug being electrically coupled thereto, said plug on said power supply conductor being pluggable into said input port in said respective sleeve such that said power supply is in electrical communication with said control circuit when said plug on said control conductor is plugged into said output port, said power supply generating said electrical impulse in accordance with said operational commands stored in said control circuit.

13. A muscle stimulation assembly being worn on a user's limbs wherein said assembly is configured to electrically stimulate the limbs for rehabilitative purposes, said assembly comprising:
a plurality of sleeves, each of said sleeves being wearable on a selected limb on a user's body, each of said sleeves being comprised of a resiliently stretchable material thereby facilitating said sleeves to compress around the selected limb, each of said sleeves having a first end, a second end and an outer wall extending therebetween, said outer wall having an inner surface and an outer surface;
a plurality of conduction arrays, each of said conduction arrays being integrated into a respective one of said sleeves, each of said conduction arrays being comprised of an electrically conductive material, said conduction array in said respective sleeve engaging skin on the limb when said respective sleeve is worn wherein said conduction array in said respective sleeve is configured to be in electrical communication with muscles in the limb when said respective sleeve is worn, each of said conduction arrays comprising:
a plurality of conductors being integrated into said outer wall of said respective sleeve wherein each of said conductors is configured to be in electrical communication with the limb when said respective sleeve is worn, said conductors being arranged in said respective sleeve to define a plurality of rings each extending around an entire circumference of said respective sleeve, said rings being spaced apart from each other and being distributed between said first end and said second end of said respective sleeve, said conductors being further arranged to extend between each of said rings such that each of said rings within said respective sleeve is in electrical communication with each other;
an input port being coupled to said outer wall of said respective sleeve, said input port being positioned proximate said first end of said sleeve, said input port being electrically coupled to an adjacent one of said rings; and
an output port being coupled to said outer wall of said respective sleeve, said output port being positioned proximate said second end of said sleeve, said output port being electrically coupled to an adjacent one of said rings; and
a control system being placed into selective electrical communication with said conduction array in said respective sleeve when said respective sleeve is worn, said control system generating an electrical impulse and communicating said electrical impulse to said conduction array in said respective sleeve, said conduction array in said respective sleeve communicating the electrical impulse to nerves in the limb wherein said control system is configured to flex muscles in the limb for therapeutic purposes, said control system comprising:
a control housing;
a control circuit being positioned within said control housing, said control circuit storing data comprising a plurality of operational commands with respect to said electrical impulse;
an electronic timer being positioned within said control housing, said electronic timer being electrically coupled to said control circuit, said electronic timer being actuatable to count down a variety of pre-determined durations of time;
a control conductor being coupled to and extending away from said control housing, said control conductor being electrically coupled to said control circuit, said control conductor having a distal end with respect to said control housing, said distal end having a control plug being electrically coupled thereto, said control plug being pluggable into said output port in said respective sleeve such that said control circuit is in electrical communication with said conductors in said respective sleeve;
a transceiver being positioned within said control housing, said transceiver being electrically coupled to said control circuit, said transceiver being in electrical communication with an extrinsic communication network;
a plurality of buttons, each of said buttons being movably coupled to said control housing, each of said buttons being electrically coupled to said control circuit, each of said buttons controlling operational parameters of said control circuit and said transceiver, said plurality of buttons including:
a plurality of timer buttons, each of said timer buttons being assigned to a respective one of said variety of durations of time associated with said electronic timer, said electronic timer counting down a respective one of said durations of time when said respective timer button is depressed, said control circuit being turned off when said electronic timer completes counting down said respective duration of time;
a hand button being, a respective one of said conduction arrays in a respective one of said sleeves being actuated when said hand button is depressed such that said respective sleeve stimulates nerves in the user's hand;
an arm button, a respective one of said conduction arrays in a respective one of said sleeves being actuated when said arm button is depressed such that said respective sleeve stimulates nerves in the user's arm;
a leg button, a respective one of said conduction arrays in a respective one of said sleeves being actuated when said leg button is depressed such that said respective sleeve stimulates nerves in the user's leg;
a foot button, a respective one of said conduction arrays in a respective one of said sleeves being actuated when said foot button is depressed such that said respective sleeve stimulates nerves in the user's foot;
a left button, each of said conduction arrays in each of said sleeves being worn on the user's left arm and left leg being actuated when said left button is depressed;
a right button, each of said conduction arrays in each of said sleeves being worn on the user's right arm and right leg being actuated when said right button is depressed; and
a power button, said control circuit being turned on or off when said power button is depressed;
a power supply housing;
a power supply being positioned within said power supply housing; and
a power supply conductor being coupled to and extending away from said power supply housing, said power supply conductor being electrically coupled to said power supply, said power supply conductor having a distal end with respect to said power supply housing, said distal end of said power supply conductor having a plug being electrically coupled thereto, said plug on said power supply conductor being pluggable into said input port in said respective sleeve such that said power supply is in electrical communication with said control circuit when said plug on said control conductor is plugged into said output port, said power supply generating said electrical impulse in accordance with said operational commands stored in said control circuit.
